(19) United States
(12) Patent Application Publication
(10) Pub. No.: US 2008/0274072 A1
(43) Pub. Date: Nov. 6, 2008

(54) Insect repellants and insecticides

(76) Inventors: John Manolas, Lake Forest, IL (US); Elli Manolas, Lake Forest, IL (US)

Correspondence Address: Michael S. Leonard;EVEREST INTELLECTUAL PROPERTY LAW GROUP, P.O. Box 708, Northbrook, IL, 60065, US

(21) Appl. No.: 11/879,828

(22) Filed: Jul. 19, 2007

Related U.S. Application Data
(60) Provisional application No. 60/927,625, filed on May 4, 2007.

(51) Int. Cl. A61L 9/013; A01P 7/04

(52) U.S. Cl. 424/76.9

(57) ABSTRACT

An insect repellant is in a sprayable gel formulation. The insect repellant is citronella based and may contain a deodorizer. An insecticide is in a sprayable gel formulation and may contain a deodorizer.

### CROSS-REFERENCE TO RELATED APPLICATIONS

This application claims priority of copending provisional application Ser. No. 60/927,625 filed May 4, 2007, incorporated herein by reference.

### BACKGROUND OF THE INVENTION

The present invention generally pertains to insect repellants, insecticides and also to deodorizers. Embodiments of the present invention provide insect repellants in formulations that are sprayable gels having an insect repellant agent, such as citronella oil. Embodiments of the present invention provide insecticides in formulations that are sprayable gels having an insecticide agent. A deodorizer may be included in the insect repellants and the insecticides. The insect repellants and insecticides may have other components as well. The present invention also pertains to methods relating to insect repellants and insecticides, including methods of using insect repellants and insecticides and methods of repelling and killing insects.

Insect repellants have been used to repel insects. Some insect repellants are citronella based. Most citronella based insect repellants have been in the form of liquid. Some citronella based insect repellants are in a powder or granular form. Citronella oil has been applied to a substrate such as clay, corn cobs, saw dust, etc. Citronella candles have also been used in attempts to repel insects. Insecticides also exist.

Sprayable gels have been around for quite some time. Sprayable gels have been used to deodorize areas such as bathrooms, garbage bins, laundry rooms, etc. However, sprayable gel insect repellants and insecticides have not existed prior to this invention.

Needs exist for new insect repellants and insecticides. Insects are numerous and frequently gather around areas where it may be undesirable to have insects. For example, trash containers tend to attract insects. Liquid insect repellants typically do not last very long. Liquid insect repellants tend to evaporate fairly quickly and thus, may not be effective for a longer period of time. Also, liquid insect repellants tend to run when applied too heavily because of their liquid form which can also reduce their effectiveness. Similarly, existing insecticides also have problems.

Accordingly, needs exist to improve insect repellants, insecticides and related methods, including methods of using insect repellants and insecticides, and methods of repelling insects and killing insects, for the reasons mentioned above and for other reasons.

### SUMMARY OF THE INVENTION

The present invention provides new insect repellants, insecticides and related methods. In an embodiment, the present invention is an insect repellant formulation containing gel and an insect repelling agent, such as citronella oil. In some other embodiments, the present invention provides insecticide formulations containing gel and an insecticide agent. The insect repellants and insecticides may be in the form of sprayable gels. The insect repellants and insecticides may also contain a deodorizer. The insect repellants and insecticides can be used, for example, in garbage dumpsters, trash bins, and other areas that are both odorous and attract insects, such as flies. Embodiments of the insect repellants and insecticides may contain water, gelling agents, citronella oil, alcohols, enzymes, and dyes or coloring agents. Additionally, other oils can be added to aid in deodorizing and repelling insects.

One unique aspect of some embodiments of the present invention is that the insect repellant is a sprayable gel formulation. Another unique aspect of some embodiments of the present invention is that the insect repellant contains citronella oil in a sprayable gel formulation. Another unique aspect of some embodiments of the present invention is that the insecticide is a sprayable gel formulation.

Features of the present invention may be described in certain embodiments. However, the present invention is broader than the described embodiments. Embodiments of the present invention may have various features and provide various advantages. Any of the features and advantages of the present invention may be desired, but, are not necessarily required to practice the present invention.

Advantages of the present invention can be to provide new insect repellants and/or insecticides.

Other advantages of the present invention can be to provide new methods related to insect repellants and/or insecticides, such as new methods of repelling insects and killing insects, and new methods of applying insect repellants and insecticides.

Other advantages of the present invention can be to provide new sprayable gel insect repellants and/or insecticides.

Further advantages of the present invention can be to provide new insect repellants and/or insecticides in sprayable gel form and which may contain a deodorizer.

Yet other advantages of the present invention can be to provide long-lasting insect repellants and/or insecticides.

Further advantages of the present invention can be to provide insect repellants and/or insecticides which can be applied to various surfaces, such as non-horizontal surfaces, and remain adhered to the surfaces for long-lasting effectiveness.

Additional features and advantages of the present invention are described in, and will be apparent from, the following Detailed Description of the Invention and the figures.

### DETAILED DESCRIPTION OF THE INVENTION

One example of a formulation according to the present invention is an insect repellant having a gel and an insect repelling agent. The insect repelling agent may be citronella oil or any other suitable insect repelling agent. The insect repellant formulation may also include components which allow the gel insect repellant to be sprayable. The insect repellant formulation may also include a deodorizing agent. Other components can be included in the insect repellant formulation as desired. The amounts of the components can be varied as desired to achieve an effective gel insect repellant.

Another example of the present invention is an insect repellant having the following formula. This insect repellant formulation is a sprayable gel formulation.

| | | |
|---|---|---|
| Water | to 100% | by volume |
| Gelling Agent | about 0.5% to about 10% | by volume |
| (Laponite RD or RDS) | | |
| Solvent (Ethanol) | about 0.1% to about 20% | by volume |
| Insect Repelling Agent | about 0.1% to about 20% | by volume |
| (Citronella oil) | | |
| Surfactant | about 0.1% to about 20% | by volume |

An example of a more specific sprayable gel formulation for an insect repellant is as follows.

| | | |
|---|---|---|
| Water | to 100% | by volume |
| Gelling Agent (Laponite RD or RDS) | about 2.5% | by volume |
| Solvent (Ethanol) | about 5.0% | by volume |
| Insect Repelling Agent | about 4.0% | by volume |
| (Citronella oil) | | |
| Surfactant | about 4.0% | by volume |

One example of suitable insect repelling agents for the formulation is citronella oil. Various other insect repelling agents can be used in the insect repellant formulation, alone or in combination. For example, essential oils having insect repelling properties can be used as the insect repelling agent. The following table lists some suitable insect repelling essential oils, and the range of amounts of each essential oil and one preferred amount of each essential oil. Of course, other insect repelling agents could be used with the present invention as well, for example without limitation, natural oils, synthetic oils, perfume blends, and other insect repelling agents. Furthermore, the insect repellant formulation can contain combinations of insect repelling agents.

| | Range % | Preferred % |
|---|---|---|
| Essential Oil | by volume | by volume |
| Eucalyptus Oil | 0.1-20% | 2% |
| Peppermint Oil | 0.1-20% | 2% |
| Clove Oil | 0.1-20% | 2% |
| Mint Oil | 0.1-20% | 2% |
| Thyme Oil | 0.1-20% | 2% |
| Tea Tree Oil | 0.1-20% | 2% |
| Neem Oil | 0.1-20% | 2% |
| Lemongrass Oil | 0.1-20% | 2% |
| Lavender Oil | 0.1-20% | 2% |
| Cedarwood Oil | 0.1-20% | 2% |
| Lemon Oil | 0.1-20% | 2% |
| Pennyroyal Oil | 0.1-20% | 2% |
| Pine Oil | 0.1-20% | 2% |
| Witch Hazel | 0.1-20% | 2% |
| Orange Oil | 0.1-20% | 2% |
| Hinoki Oil (White Cedar) | 0.1-20% | 2% |
| Geranium Oil | 0.1-20% | 2% |
| Coconut Oil | 0.1-20% | 2% |
| Bergamot Oil | 0.1-20% | 2% |
| Cinnamon Oil | 0.1-20% | 2% |
| Corn Oil | 0.1-20% | 2% |
| Cottonseed Oil | 0.1-20% | 2% |
| Garlic Oil | 0.1-20% | 2% |
| Linseed Oil | 0.1-20% | 2% |
| Rosemary Oil | 0.1-20% | 2% |
| Sesame Oil | 0.1-20% | 2% |
| Soybean Oil | 0.1-20% | 2% |

One example of suitable solvents is alcohol. Some examples of suitable alcohols are ethanol, methanol and isopropanol. Some other examples of suitable solvents include propylene glycol and hexelene glycol. Of course, other solvents can be used with the present invention as well. One feature of the solvent can be to provide volatility to the insect repelling agent, such as citronella oil, so that the insect repelling agent can be effectively dispersed in the air.

The surfactant helps place the oil based insect repelling agent in the water phase. A wide variety of surfactants can be used with the present invention. Some examples of suitable surfactants are non-ionic surfactants, ampohoteric surfactants, cationic surfactants and anionic surfactants. One example of a suitable surfactant is Tergitol NP-9.

Two examples of a suitable gelling agent are Laponite RD or Laponite RDS. The Laponite forms a gel when mixed with water. Other gelling agents can be used with the present invention as well.

The water in the formulation is a carrier. Other suitable carriers could be used if desired.

Formulations according to the present invention can include various other components if desired. For example, deodorizers can be added to the formulation. Deodorizers can include, for example, enzymes, bacteria and blends of enzymes and bacteria. Some examples of suitable enzymes include protease, lipase, amylase, cellulose, single strain enzymes and enzyme blends.

Coloring agents or dyes can be added to the formulations to provide colors, for example to color code different insect repellant products.

In another embodiment, the present invention provides insecticides, i.e. insect killing formulations. The insecticide formulations can have basically the same formulations as the insect repellant formulations, except the insect repelling agent is replaced with an insect killing agent. Alternatively, formulations according to the present invention can include both insect repelling agents and insect killing agents. The following table lists some suitable insect killing agents and the range of amounts of each insect killing agent and one preferred amount of each insect killing agent. Of course, other insect killing agents could be used with the present invention as well.

| | Range % | Preferred % |
|---|---|---|
| Insecticide | By volume | By volume |
| Pyrethrin | 0.1-5% | 0.2% |
| Permithrin | 0.1-5% | 0.2% |
| Tetramethrin | 0.1-5% | 0.2% |
| Resmethrin | 0.1-5% | 0.2% |

An example of making or mixing an insect repellant formulation according to the present invention will now be described. The insect repellant formulation is mixed by adding the gelling agent (Laponite) to the water and mixing until dispersed. The ethanol, citronella oil and surfactant are pre-mixed in a separate container. The oils are then added to the water slowly with agitation. Dyes and enzymes, if desired, can be added to the water phase prior to the addition of the oils. The mixed insect repellant formulation can be loaded into containers, for example spray bottles for use. Embodiments of the insecticide formulations can be similarly mixed.

Embodiments of the insect repellant formulations can be used as follows. Sprayable gel insect repellant formulations can be sprayed from a trigger sprayer (similar to spray bottle containing glass cleaner) onto any desired surface. The insect repellant formulation will repel insects, such as flies. Embodiments of the insect repellant formulation which include a deodorizer will also deodorize the area to which the formulation is applied. Embodiments of the insect repellant formulations which are gel formulations, but not sprayable, are also applied to the desired surface. An instrument can be used, if desired, to apply the non-sprayable gel formulation. Embodiments of the insecticide formulations can be similarly used.

Embodiments of the insect repellant formulations and the insecticide formulations can be stored in and applied from many different types of containers. In addition to spray bottles having trigger sprayers, squeeze bottles having dispensing nozzles can be used, for example. The formulations can be applied by squeezing the bottle and squirting the formulations through a nozzle, such as nozzles used in containers having disc tops, flip tops, snap tops, push/pull tops and other tops. Yet another example of dispensing formulations of the present invention is to contain the formulations in aerosol spray containers and dispensing the formulations by aerosol propellant.

An advantage of this invention can be the vertical cling to surfaces allowing for extended cling time allowing for longer effectiveness of the insect repelling agent and/or the insect killing agent. The present invention can effectively repel insects and/or kill insects and/or deodorize.

It should be understood that various changes and modifications to the presently preferred embodiments described herein will be apparent to those skilled in the art. Such changes and modifications can be made without departing from the spirit and scope of the present invention and without diminishing its intended advantages.

The invention is claimed as follows:

1. An insect repellant formulation, comprising a gel and an insect repelling agent.

2. The insect repellant formulation of claim 1, further comprising a deodorizer.

3. The insect repellant formulation of claim 1, wherein the insect repellant formulation is a sprayable gel formulation.

4. The insect repellant formulation of claim 2, wherein the insect repellant formulation is a sprayable gel formulation.

5. The insect repellant formulation of claim 1, comprising:
water to 100% by volume;
a gelling agent at about 0.5% to about 10% by volume;
a solvent at about 0.1% to about 20% by volume;
the insect repelling agent at about 0.1% to about 20% by volume; and
a surfactant at about 0.1% to about 20% by volume.

6. The insect repellant formulation of claim 5, comprising:
the water to 100% by volume;
the gelling agent at about 2.5% by volume;
the solvent at about 5% by volume;
the insect repelling agent at about 4% by volume; and
the surfactant at about 4% by volume.

7. The insect repellant formulation of claim 5, wherein the insect repelling agent is selected from the group consisting of citronella oil, eucalyptus oil, peppermint oil, clove oil, mint oil, thyme oil, tea tree oil, neem oil, lemongrass oil, lavender oil, cedarwood oil, lemon oil, pennyroyal oil, pine oil, witch hazel, orange oil, hinoki oil, geranium oil, coconut oil, bergamot oil, cinnamon oil, corn oil, cottonseed oil, garlic oil, linseed oil, rosemary oil, sesame oil, soybean oil, essential oils, natural oils, synthetic oils, perfume blends and combinations thereof.

8. The insect repellant formulation of claim 6, wherein the insect repelling agent is selected from the group consisting of citronella oil, eucalyptus oil, peppermint oil, clove oil, mint oil, thyme oil, tea tree oil, neem oil, lemongrass oil, lavender oil, cedarwood oil, lemon oil, pennyroyal oil, pine oil, witch hazel, orange oil, hinoki oil, geranium oil, coconut oil, bergamot oil, cinnamon oil, corn oil, cottonseed oil, garlic oil, linseed oil, rosemary oil, sesame oil, soybean oil, essential oils, natural oils, synthetic oils, perfume blends and combinations thereof.

9. The insect repellant formulation of claim 5, wherein the solvent is selected from the group consisting of alcohol, ethanol, methanol, isopropanol, propylene glycol, hexelene glycol and combinations thereof.

10. The insect repellant formulation of claim 6, wherein the solvent is selected from the group consisting of alcohol, ethanol, methanol, isopropanol, propylene glycol, hexelene glycol and combinations thereof.

11. The insect repellant formulation of claim 5, wherein the surfactant is selected from the group consisting of non-ionic surfactants, ampohoteric surfactants, cationic surfactants, anionic surfactants and combinations thereof.

12. The insect repellant formulation of claim 6, wherein the surfactant is selected from the group consisting of non-ionic surfactants, ampohoteric surfactants, cationic surfactants, anionic surfactants and combinations thereof.

13. The insect repellant formulation of claim 5, further comprising a deodorizer.

14. The insect repellant formulation of claim 13, wherein the deodorizer is selected from the group consisting of enzymes, bacteria, blends of enzymes and bacteria, protease, lipase, amylase, cellulose, single strain enzymes, enzyme blends and combinations thereof.

15. The insect repellant formulation of claim 6, further comprising a deodorizer.

16. The insect repellant formulation of claim 15, wherein the deodorizer is selected from the group consisting of enzymes, bacteria, blends of enzymes and bacteria, protease, lipase, amylase, cellulose, single strain enzymes, enzyme blends and combinations thereof.

17. The insect repellant formulation of claim 1, further comprising an insect killing agent.

18. An insecticide formulation, comprising a gel and an insect killing agent.

19. The insecticide formulation of claim 18, further comprising a deodorizer.

20. The insecticide formulation of claim 18, wherein the insecticide formulation is a sprayable gel formulation.

21. The insect repellant formulation of claim 19, wherein the insecticide formulation is a sprayable gel formulation.

22. The insecticide formulation of claim 18, comprising:
water to 100% by volume;
a gelling agent at about 0.5% to about 10% by volume;
a solvent at about 0.1% to about 20% by volume;
the insect killing agent at about 0.1% to about 5% by volume; and
a surfactant at about 0.1% to about 20% by volume.

23. The insecticide formulation of claim 22, comprising:
the water to 100% by volume;
the gelling agent at about 2.5% by volume;
the solvent at about 5% by volume;
the insect killing agent at about 0.2% by volume; and
the surfactant at about 4% by volume.

24. The insecticide formulation of claim 22, wherein the insect killing agent is selected from the group consisting of pyrethrin, permithrin, tetramethrin, resmethrin and combinations thereof.

25. The insecticide formulation of claim 23, wherein the insect killing agent is selected from the group consisting of pyrethrin, permithrin, tetramethrin, resmethrin and combinations thereof.

26. The insecticide formulation of claim 22, wherein the solvent is selected from the group consisting of alcohol, ethanol, methanol, isopropanol, propylene glycol, hexelene glycol and combinations thereof.

27. The insecticide formulation of claim 23, wherein the solvent is selected from the group consisting of alcohol, ethanol, methanol, isopropanol, propylene glycol, hexelene glycol and combinations thereof.

28. The insecticide formulation of claim 22, wherein the surfactant is selected from the group consisting of non-ionic surfactants, ampohoteric surfactants, cationic surfactants, anionic surfactants and combinations thereof.

29. The insecticide formulation of claim 23, wherein the surfactant is selected from the group consisting of non-ionic surfactants, ampohoteric surfactants, cationic surfactants, anionic surfactants and combinations thereof.

30. The insecticide formulation of claim 22, further comprising a deodorizer.

31. The insecticide formulation of claim 30, wherein the deodorizer is selected from the group consisting of enzymes, bacteria, blends of enzymes and bacteria, protease, lipase, amylase, cellulose, single strain enzymes, enzyme blends and combinations thereof.

32. The insecticide formulation of claim 23, further comprising a deodorizer.

33. The insecticide formulation of claim 32, wherein the deodorizer is selected from the group consisting of enzymes, bacteria, blends of enzymes and bacteria, protease, lipase, amylase, cellulose, single strain enzymes, enzyme blends and combinations thereof.

34. A method of making an insect-affecting formulation, comprising:
mixing a gelling agent and water into a gel/water mixture; and
mixing an inset-affecting agent with the gel/water mixture.

35. The method of making an insect-affecting formulation of claim 34, wherein mixing an inset-affecting agent with the gel/water mixture comprises mixing an insect repelling agent into the formulation.

36. The method of making an insect-affecting formulation of claim 34, wherein mixing an inset-affecting agent with the gel/water mixture comprises mixing an insect killing agent into the formulation.
